(19) United States
(12) Patent Application Publication
(10) Pub. No.: US 2017/0000563 A1
(43) Pub. Date: Jan. 5, 2017

(54) METHOD, APPARATUS AND SYSTEM FOR SIMULATING FORCE INTERACTION BETWEEN BONE DRILL AND SKELETON

(71) Applicant: SHENZHEN INSTITUTES OF ADVANCED TECHNOLOGY CHINSE ACADEMY OF SCIENCES, Shenzhen (CN)

(72) Inventors: Qiong WANG, Shenzhen (CN); Hui CHEN, Shenzhen (CN); Wen WU, Shenzhen (CN); Jing QIN, Shenzhen (CN); Haiyang JIN, Shenzhen (CN); Ping' an WANG, Shenzhen (CN)

(21) Appl. No.: 15/039,771

(22) PCT Filed: Dec. 4, 2013
(86) PCT No.: PCT/CN2013/088509

§ 371 Date: May 26, 2016

(30) Foreign Application Priority Data
Nov. 26, 2013 (CN) 201310608357.3

(51) Int. Cl. A61B 34/10; A61C 3/02

(52) U.S. Cl. CPC A61B 34/10; A61C 3/02; A61B 2034/102; A61B 2034/105; A61B 2034/104

(57) ABSTRACT

A method, apparatus and system for simulating force interaction between a bone drill and a skeleton applicable to the field of force interaction. The method comprises: detecting whether a collision takes place between a bone drill module (2) and a skeleton module (3) in real time; when a collision takes place, acquiring a movement speed and an autorotation speed of each collision point before the collision; calculating a movement speed and an autorotation speed of each collision point after the collision; removing a collision point having a separation speed with respect to the skeleton module (3) after the collision; calculating a resistance force and a frictional force on a collision point that is not removed at the time of collision according to the movement speeds and autorotation speeds before and after the collision and a method based on impulse theory; and synthesizing resistance forces and frictional forces of all collision points that are not removed into a resultant force to output same to a force feedback device. By using a method based on impulse theory to calculate a resistance force and a frictional force on a collision point that is not removed at the time of collision, force differences brought about by force interaction, such as grinding, among different bone drills and skeletons can be effectively reflected.

### TECHNICAL FIELD

The present application relates to the technical field of force interaction, and more particularly, relates to a method, apparatus and system for simulating force interaction between a bone drill and a skeleton.

### BACKGROUND

A bone grinding operation is the most commonly used operation technique in various orthopedic surgeries, and is mainly used to remove a part of sclerotin to expose an operative area, or shape a diaphysis, for example, grinding a bone spur or an osteophyte in a surgery for protrusion of intervertebral disc; using a bone drill to create a slippy groove so as to replace a worn acetabular cup in a hip joint replacement surgery; shaping a path on a skull by grinding to remove a brain tumor, and so on. Most of these types of surgeries are irreversible, and thus any error in the surgeries may led to a serious injury to a patient; for example, if the control is not well performed in the process of bone grinding, friable tissues, such as nerves, blood vessels, and so on, are very likely to be injured. Therefore, young doctors need to experience hard training for long time before they can carry out operations accurately and safely. In traditional training methods, the doctors can only exercise on plastic human body models, animals, dead bodies, and patients, however, these methods usually have various problems, for example, these methods may be unreal, expensive, unable to be reused, and may bring patients into troubles.

A surgery simulation system based on VR (Virtual Reality), as a safe and reliable surgery training apparatus, has attracted more and more attention. How to simulate force interaction between a bone drill model and a skeleton model effectively and as realistic as possible is a key problem to be solved in the surgery simulation system. In the existing methods for simulating force interaction between a bone drill model and a skeleton model, a commonly used method is simulating skeleton grinding based on a metal grinding theory and according to a metal cutting mechanism; however, a physical property of metal is quite different from that of skeletons, force differences brought by force interaction, such as grinding between a bone drill and a skeleton of different materials, can't be reflected.

To sum up, the existing methods for simulating force interaction between a bone drill model and a skeleton model can't reflect force differences brought by force interaction between a bone drill and a skeleton of different materials.

### Technical Problem

Embodiments of the present invention aim at providing a method for simulating force interaction between a bone drill and a skeleton, for the purpose of solving the problem that an existing method for simulating force interaction between a bone drill model and a skeleton model can't reflect force differences brought by force interaction between the bone drill and the skeleton of different materials.

### Technical Solution

The embodiments of the present invention are achieved as follows. A method for simulating force interaction between a bone drill and a skeleton, comprising:

detecting whether a collision takes place between a bone drill model and a skeleton model in real time;

when the collision takes place, acquiring a movement speed and an autorotation speed of each collision point before the collision;

calculating a movement speed and an autorotation speed of each collision point after the collision according to the impulse theory, the Newton's impact law, the Coulomb's law, and the movement speed and the autorotation speed of each collision point before the collision;

removing a collision point having a separation speed with respect to the skeleton model after the collision;

calculating a resistance force and a friction force on a collision point that is not removed at the time of collision according to the movement speeds and autorotation speeds before and after the collision and using a method based on the impulse theory; and

synthesizing resistance forces and friction forces of all the collision points that are not removed into a resultant force to output the resultant force to a force feedback device.

Another embodiment of the present invention further provides an apparatus for simulating force interaction between a bone drill and a skeleton, the apparatus comprises:

a collision detecting unit configured for detecting whether a collision takes place between a bone drill model and a skeleton model in real time;

a speed obtaining unit configured for when the collision takes place, obtaining a movement speed and an autorotation speed of each collision point before the collision;

a speed calculating unit configured for calculating the movement speed and the autorotation speed of each collision point after the collision according to the impulse theory, the Newton's impact law, the Coulomb's law, and the movement speed and the autorotation speed of each collision point before the collision;

a removing unit configured for removing a collision point having a separation speed with respect to the skeleton model after the collision;

a first calculating unit configured for calculating a resistance force and a friction force on a collision point that is not removed at the time of collision according to movement speeds and autorotation speeds before and after the collision and using a method based on the impulse theory; and

a force synthesizing unit configured for synthesizing resistance forces and friction forces of all the collision points that are not removed into a resultant force to output the resultant force to a force feedback device.

Another embodiment of the present invention further provides a virtual surgical system, the virtual surgical system comprises the aforesaid apparatus for simulating force interaction between a bone drill and a skeleton.

### Beneficial Effects

Compared with the prior art, advantageous effects of the embodiments of the present invention is that: by using the method based on the impulse theory to calculate the resistance force and the friction force on the collision point that is not removed at the time of collision, during the process of calculation, a hybrid restitution coefficient e can be fully reflected in the resistance forces and the friction forces, thereby well reflecting the material properties of the bone drill and the skeleton, and thus force differences brought by force interaction, such as grinding between a bone drill and a skeleton of different materials, can be effectively reflected.

### BRIEF DESCRIPTION OF THE DRAWINGS

FIG. 1 illustrates a flow chart of a method for simulating force interaction between a bone drill and a skeleton provided by one embodiment of the present invention;

FIG. 2 illustrates a schematic view of a bone drill model provided by one embodiment of the present invention;

FIG. 3 illustrates a schematic view of a skeleton model provided by one embodiment of the present invention;

FIG. 4 illustrates a schematic diagram of collision interaction before a collision in the method for simulating force interaction between a bone drill and a skeleton provided by the embodiment of the present invention;

FIG. 5 illustrates a schematic diagram of collision interaction after the collision in the method for simulating force interaction between a bone drill and a skeleton provided by the embodiment of the present invention;

FIG. 6 illustrates a schematic diagram of a friction cone provided by one embodiment of the present invention;

FIG. 7 illustrates a schematic diagram of a vibration model provided by one embodiment of the present invention.

FIG. 8 illustrates a logic structure schematic diagram of an apparatus for simulating force interaction between a bone drill and a skeleton provided by one embodiment of the present invention.

### DETAILED DESCRIPTION OF THE PREFERRED EMBODIMENT

In order to make the purpose, technical solutions and advantages of the present invention be clearer and more understandable, the present invention will be described in detail with reference to accompanying drawings and embodiments; it should be understood that the specific embodiments described herein are only used to explain the present invention, rather than limiting the present invention.

An implementation scheme of one embodiment of the present invention is as follows:

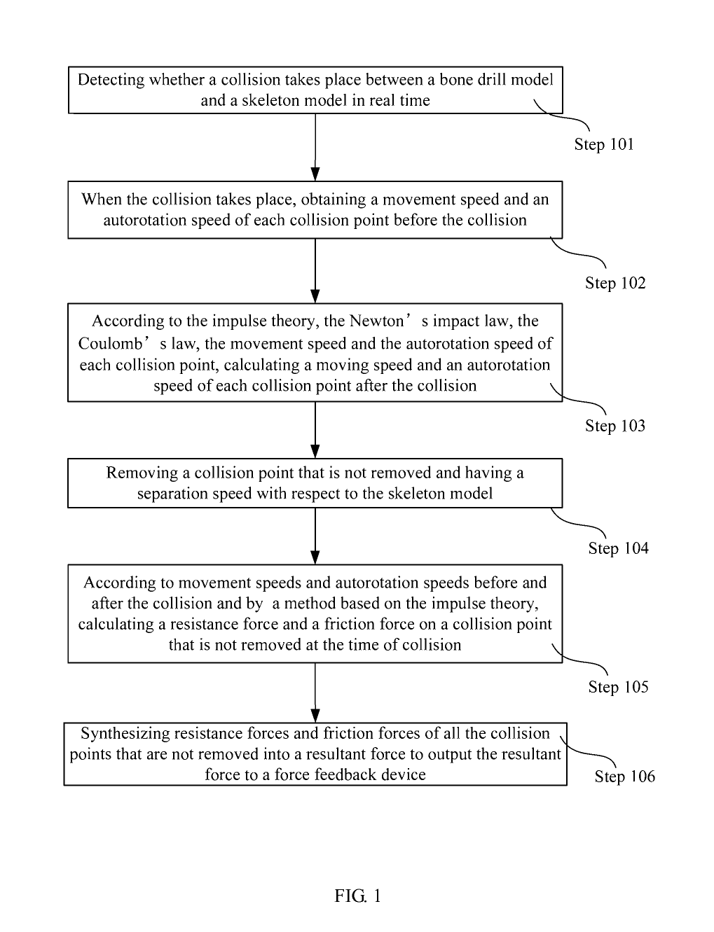

please refer to FIG. 1, the embodiment of the present invention provides a method for simulating force-sensing interaction between a bone drill and a skeleton, the method is applied in a computer terminal, and the method comprises:

101, detecting whether a collision takes place between a bone drill model and a skeleton model in real time;

in the embodiment of the present invention, this step comprises:

uniformly distributing a predetermined number of discrete points on a cutting edge of the bone drill model in advance;

connecting a line segment between each of the discrete points and a center point O of the bone drill model;

detecting whether the line segment and a triangle surface patch of the bone drill model share a cross point in real time; if yes, determining that the collision has taken place between the bone drill model and the skeleton model, and recording the discrete point at which the collision has taken place as a collision point; if no, determining that the collision doesn't take place between the bone drill model and the skeleton model. Correspondingly, the collision points in the following steps 102, 103, 104, 105, 106 are specifically the discrete points at which the collision has taken place.

In the embodiment of the present invention, before the step 101, the method further comprises the following step:

establishing the bone drill model and the skeleton model in advance. A surface of the skeleton model is comprised of a plurality of small triangle surface patches that are combined together.

Step 102, when the collision has taken place, obtaining a movement speed and an autorotation speed of each of the collision points before the collision;

In the embodiment of the present invention, by reading an operation speed of a force feedback device that is connected with the computer terminal, the movement speed before the collision can be obtained.

Specifically, the force feedback device in the embodiment of the present invention simulates a form of a bone grinding surgical tool, and appears as similar as possible in a specific application, thereby providing a user with a verisimilar experience. When the user operates the force feedback device, the force feedback device will be driven to move; in the method of the present invention, when the collision has taken place between the bone drill model and the skeleton model, the movement speed of the force feedback device being driven before the moment of collision can be read by the computer terminal and used as the movement speed of the collision point before the collision. The autorotation speed of the force feedback device is the autorotation speed of the bone drill model, and thus it can be correspondingly set as needed in the computer terminal.

Step 103, according to the impulse theory, the Newton's impact law, the Coulomb's law, and the movement speed \vec{V}ᵢ and the autorotation speed \vec{ω} of each of the collision points before the collison, calculating the movement speed \vec{U}ᵢ and the autorotation speed \vec{ω}′ of each of the collision points after the collision. The calculating formulas can be the following formulas:

\vec{U}ᵢ=\vec{V}ᵢ+\vec{P}ᵢ/M

\vec{ω}′=\vec{ω}+J⁻¹(\vec{r}ᵢ×\vec{P}ᵢ).

Wherein, \vec{P}ᵢ represents a total impulse, J represents an inertia tensor, \vec{r}ᵢ represents a momentum that directs from 0 to a collision point i, M is a quality of a round head part of the hone drill. Relevant principles and calculation for each of these parameters are described in detail in a specific calculation process which will be described later.

Step 104, removing any of the collision points having a separation speed with respect to the skeleton model after the collision;

in the embodiment of the present invention, a collision point is a discrete point at which the collision has taken place. Each discrete point and a cross point corresponding to the discrete point are called as a collision point pair. The step 104 comprises:

arranging the discrete points at which the collision has taken place in an descending order according to the distances between the collision point pairs, thereby generating a list;

traversing all the discrete points at which the collision has taken place in the list, and judging whether each of the discrete points after the collision has a separation speed with respect to the skeleton model;

if yes, removing the discrete point having the separation speed with respect to the skeleton model from the list.

Step 105, according to the movement speed and the autorotation speed before and after the collision, and by a method based on the impulse theory, calculating a resistance force and a friction force of each of the collision points that is not removed at the time of collision.

Step 106, synthesizing the resistance forces and the friction forces of all the collision points that are not removed into a resultant force to output the resultant force to the force feedback device.

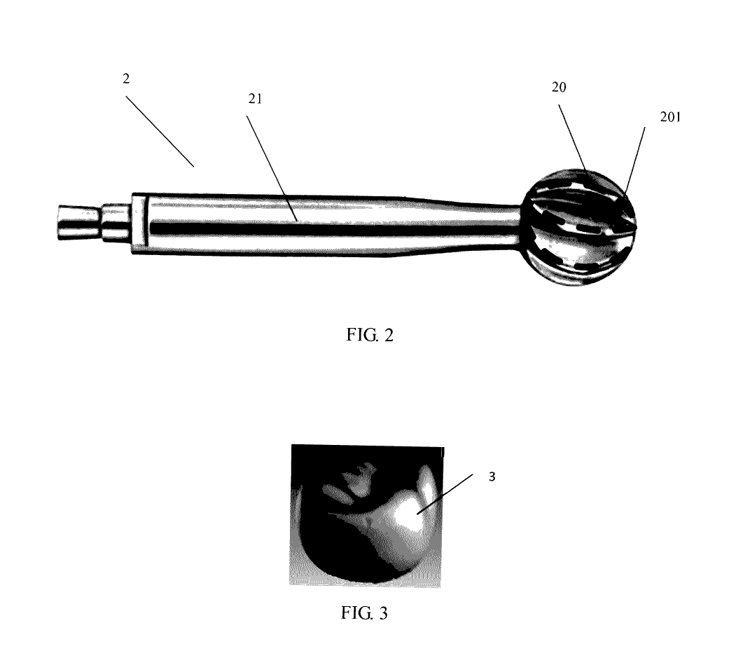

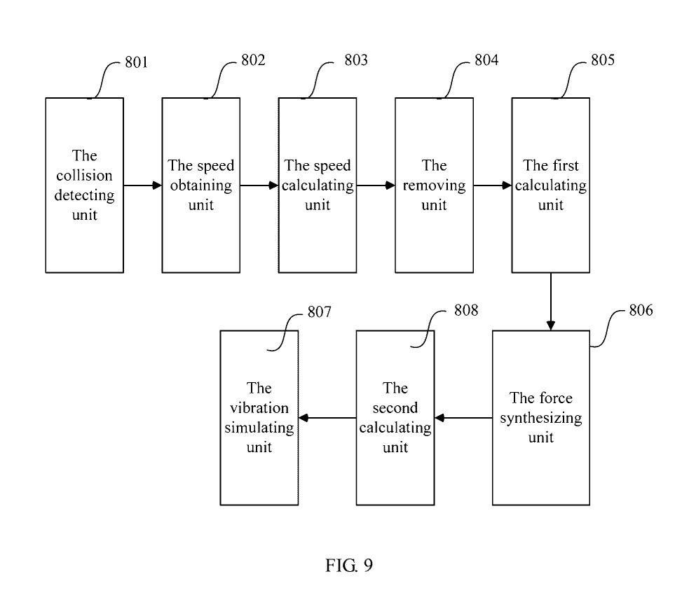

Please refer to FIG. 2 and FIG. 7, in one embodiment of the present invention, the bone drill model 2 comprises a round head 20 and a long shaft 21; the method further comprises:

simulating the long shaft 21 into a straight rod of which a distal end is connected to an electric drive device through a spring Kx in an X-axis direction, a spring Ky in a Y-axis direction, and a spring Kz in a Z-axis direction, so that vibrations of the long shaft 21 in a horizontal direction and an axial direction are simulated. A vibration model is shown in FIG. 7;

calculating a vibration force applied on the long shaft 21 when the long shaft 21 is vibrated;

the step of synthesizing the resistance forces and the friction forces of all the collision forces into the resultant force specifically includes:

synthesizing the resistance forces and the friction forces of all the collision points that are not removed, and the vibration force applied on the long shaft 21 into a resultant force.

In the embodiment of the present invention, the resistance force is

$${\vec{f}}_{{n}_{i}} = \frac{{\vec{P}}_{{n}_{i}}}{{t}_{1} - {t}_{0}} ;$$

the friction force comprises a static friction force and a dynamic friction force: the static friction force is

$${\vec{f}}_{{τ}_{i}} = \frac{{\vec{P}}_{i} - {\vec{P}}_{{n}_{i}}}{{t}_{1} - {t}_{0}} ;$$

the dynamic friction force is

$${\vec{f}}_{{τ}_{i}} = \frac{{\vec{P}}_{i} - {\vec{P}}_{{n}_{i}}}{{t}_{1} - {t}_{0}} ;$$

the vibration force is \vec{f}_{vib}=2MₛΔ\vec{d}(t)/(t₁−t₀)²; a hybrid coefficient of restitution is

$$e = \sqrt{( \frac{{e}_{tool}^{2} ( 1 - {v}_{tool}^{2} )}{{E}_{tool}} + \frac{{e}_{bone}^{2} ( 1 - {v}_{bone}^{2} )}{{E}_{bone}} ) E} ,$$

wherein, E=EₜₒₒₗE_{bone}/(Eₜₒₒₗ+E_{bone});

wherein, a unit vector directed from the discrete point at which the collision has taken place to a corresponding cross point is recorded as \vec{n}ᵢ; \vec{P}ₙ_{ᵢ} is an impulse generated in the direction of the unit vector \vec{n}ᵢ of the discrete point at which the collision has taken place and in a time interval from a time t₀ to a time t₁ by the resistance force \vec{f}ₙ_{ᵢ}, \vec{P}ᵢ is a total impulse applied on the discrete point at which the collision has taken place during the process of collision lasting from the time t₀ to the time t₁; \vec{P}ₙ_{ᵢ} and \vec{P}ᵢ are calculated specifically according to the obtained movement speeds and autorotation speeds of the discrete point at which the collision has taken place before and after the collision, and according to the Newton's collision law, the impulse theory and the theorem of momentum, Mₛ represents a mass of the long shaft, Δ\vec{d}(t) represents a vibration displacement, Δ\vec{d}(t) is calculated specifically by a transformation function matrix and a fourth-order Runge-Kutta numerical value method; Eₜₒₒₗ and E_{bone} are respectively Young's modulus of the bone drill model and the skeleton model, vₜₒₒₗ and v_{bone} are respectively Poisson's coefficients of the bone drill model and the skeleton model, eₜₒₒₗ and e_{bone} are respectively coefficients of restitution of the bone drill model and the skeleton model, and E is an effective Young's modulus in the process of collision.

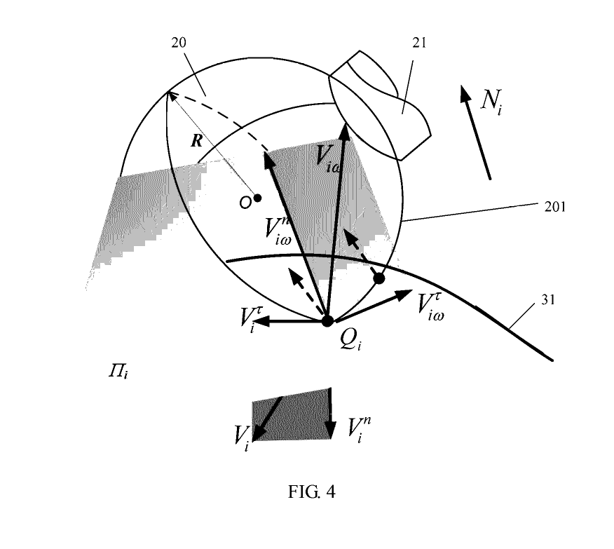

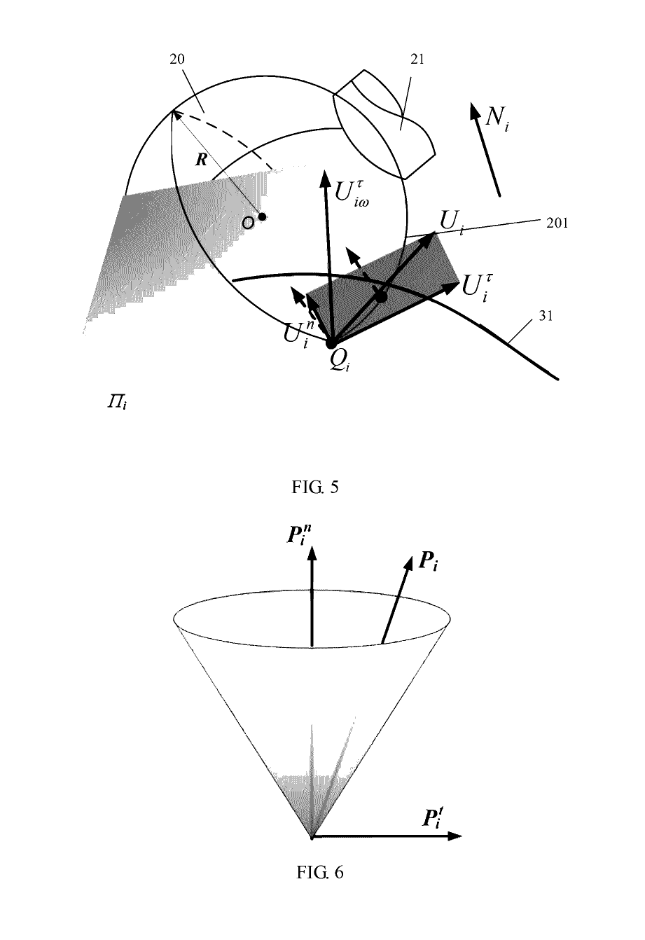

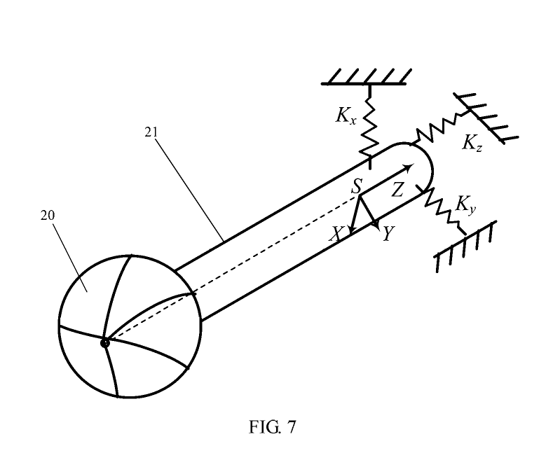

In the embodiment of the present invention, by a method of Ray-collision detection, whether the collision has taken place between the bone drill model and the skeleton model or not is detected. Please refer to FIGS. 2-7, the embodiment of the present invention averagely distributes a predetermined number of discrete points on a cutting edge 201 of the round head 20 of the bone drill model 2, and connects a line segment between each of the discrete points and the center point O of the bone drill model 2; when the bone drill model 2 contacts the skeleton model 3, whether the line segment and the triangle surface patch 31 of the skeleton model 3 share the cross point or not is detected in real time; if yes, it is determined that the collision has taken place between the bone drill model 2 and the skeleton model 3, and each discrete point at which the collision has taken place is recorded as a collision point; if no, it is determined that the collision doesn't take place between the bone drill model 2 and the skeleton model 3. In the embodiment of the present invention, each collision point and a cross point that corresponds to the collision point are also called as a collision point pair. As shown in FIG. 4 and FIG. 5, a discrete point on the bone drill model 2 is recorded as Qᵢ, a vector quantity that directs from Qᵢ to the cross point s recorded as nᵢ, a contact surface Π, is recorded as passing through Qᵢ and being perpendicular to nᵢ. When the collision takes place on the discrete point Qᵢ, the discrete point Qᵢ at which the collision has taken place is recorded as a collision point i. Speeds of the discrete point Qᵢ before and after the collision are distinguished by a time point at the moment of the collision; FIG. 4 illustrates a movement state of the discrete point Qᵢ before the collission takes place between the discrete point Qᵢ and the skeleton model 3, FIG. 5 illustrates a state of the discrete point Qᵢ after the collision has taken place between the discrete point Qᵢ and the skeleton model 3. The speeds before and after the collision are decomposed, and speed viarables before and after the decomposition and mutual relationships among these speed variables are shown in following table 1:

Speed variables table 1.

| Variable | |
|---|---|
| label | Meaning |
| \vec{ω} | An angular speed of the bone drill model before the |
| | collision |
| \vec{V}ᵢ | A movement speed of Qᵢ before the collision |
| \vec{V}ₙ_{ᵢ} | A vertical component of \vec{V}ᵢ on Πᵢ |
| \vec{V}_{τ}_{ᵢ} | A horizontal component of \vec{V}ᵢ on Πᵢ |
| \vec{V}_{ωi} | A linear speed on Qᵢ caused by the angular speed before |
| | the collision, \vec{V}_{ωi} = \vec{ω} × \vec{r}ᵢ′, \vec{r}ᵢ′ is a unit vector that directs |
| | from a cross point of Qᵢ and a rotating shaft of the bone |
| | drill model to Qᵢ |
| \vec{V}_{ωn}_{ᵢ} | A vertical component of \vec{V}_{ωi} on Πᵢ |
| \vec{V}_{ωτ}_{ᵢ} | A horizontal component of \vec{V}_{ωi} on Πᵢ |
| \vec{V}_{cn}_{ᵢ} | \vec{V}ₙ_{ᵢ} + \vec{V}_{ωn}_{ᵢ}, the total of the vertical components on Qᵢ |
| | before the collision |
| \vec{V}_{cτ}_{ᵢ} | \vec{V}_{τ}_{ᵢ} + \vec{V}_{ωτ}_{ᵢ}, the total of the horizontal components on Qᵢ |
| | before the collision |
| \vec{ω}′ | The angular speed of the bone drill after the collision |
| \vec{U}ᵢ | The movement speed of Qᵢ after the collision |
| \vec{U}ₙ_{ᵢ} | A vertical component of \vec{U}ᵢ on Πᵢ |
| \vec{U}_{τ}_{ᵢ} | A horizontal component of \vec{U}ᵢ on Πᵢ |
| \vec{U}_{ωi} | A linear speed on Qᵢ caused by the angular speed after the |
| | collision |
| \vec{U}_{ωn}_{ᵢ} | A vertical component of \vec{U}_{ωi} on Πᵢ |
| \vec{U}_{ωτ}_{ᵢ} | A horizontal component of \vec{U}_{ωi} on Πᵢ |
| \vec{U}_{cn}_{ᵢ} | \vec{U}ₙ_{ᵢ} + \vec{U}_{ωn}_{ᵢ}, the total of vertical components on Qᵢ after the |
| | collision |
| \vec{U}_{cτ}_{ᵢ} | \vec{U}_{τ}_{ᵢ} + \vec{U}_{ωτ}_{ᵢ}, the total of horizontal components on Qᵢ after |
| | the collision |

A detailed calculation process of the resistance forces, the friction forces, the resultant force after removing, the vibration forces, and the hybrid coefficient of restitution e will be introduced in detail below according to the table 1, specifically as follows:

One, the calculation process of the resistance force.

In the embodiment of the present invention, the resistance force is a force applied vertically on a collision plane, and its main function is preventing the bone drill model 2 from entering the interior of the skeleton model 2 and cutting off sclerotin. According to momentum theorem, the resistance force on the collision point i can be represented by the following formula:

M(\vec{U}ₙ_{ᵢ}−\vec{V}ₙ_{ᵢ})=∫ₜ_{₀}^{t}^{₁}\vec{f}ₙ_{ᵢ}(t)dt=\vec{P}ₙ_{ᵢ} (1)

in formula (1), M represents the mass of the round head of the bone drill, \vec{f}ₙ_{ᵢ} represents the resistance force applied on the collision point i, \vec{P}ₙ_{ᵢ} is the impulse generated by the force \vec{f}ₙ_{ᵢ} applied on the skeleton model during a time interval from t₀ to t₁. At the moment of collision, supposing that the skeleton model 3 is absolutely rest, a relative speed of the bone drill model 2 and the skeleton model 3 at the collision point is equal to a speed of the bone drill model 2 at the collision point.

According to the Newton's impact law, the relative speed of the collision point in a vertical direction after the collision can be calculated by the following formula:

\vec{U}_{cn}_{ᵢ}=−e\vec{V}_{cn}_{ᵢ}=−e(\vec{V}ₙ_{ᵢ}+\vec{V}_{ωn}_{ᵢ}) (2)

Wherein, e is the hybrid coefficient of restitution, which can be determined by material properties of two collision objects, and can be obtained by the following formula (18).

According to the impulse theory, the impulse of the collision point in the vertical direction can be obtained by the following formula:

\vec{P}ₙ_{ᵢ}=Kᵢ⁻¹(\vec{U}_{cn}_{ᵢ}−\vec{V}_{cn}_{ᵢ}) (3)

Wherein, Kᵢ is a three-dimensional collision matrix, and can be obtained by the following formula:

$${K}_{i} = \frac{1}{M} I + {r}_{i}^{*} {J}^{- 1} {r}_{i}^{*} \qquad ( 4 )$$

Wherein, I represents a three-dimensional unit matrix, r*ᵢ represents a cross product matrix of a momentum \vec{r}ᵢ that directs from 0 to the collision point i, J represents the inertia tensor. Wherein, \vec{r}*ᵢ is the cross product matrix of the \vec{r}ᵢ=[rix,riy,riz] and can be expressed as

$${r}_{i}^{*} = [ \begin{matrix}0 & - {r}_{iz} & {r}_{iy} \\ {r}_{iz} & 0 & - {r}_{ix} \\ - {r}_{iy} & {r}_{ix} & 0\end{matrix} ] ;$$

finally, the resistance force applied on the collision point can be obtained by the following formula:

$${\vec{f}}_{{n}_{i}} = \frac{{\vec{P}}_{{n}_{i}}}{{t}_{1} - {t}_{0}} \qquad ( 5 )$$

Two, the calculation process of the friction force is as follows.

The friction force in the embodiment of the present invention is in accordance with the theorem of momentum, the friction force at the collision point i can be expressed as the following formula:

M(\vec{U}_{τ}_{ᵢ}−\vec{V}_{τ}_{ᵢ})=∫ₜ_{₀}^{t}^{₁}\vec{f}_{τ}_{ᵢ}(t)dt=\vec{P}_{τ}_{ᵢ} (6)

Wherein, \vec{f}_{τ}_{ᵢ} is the friction force, \vec{P}_{τ}_{ᵢ} is the impulse generated by \vec{f}_{τ}_{ᵢ} applied on the skeleton model during the time interval lasting from t₀ to t₁. \vec{P}_{τ}_{ᵢ} and \vec{f}_{τ}_{ᵢ} are horizontal to the collision plane of the collision point i. The friction forces in the embodiment of the present invention comprise two modes, i.e., a static friction force mode and a dynamic friction force mode, which are determined by the direction of the total impulse \vec{P}ᵢ. At the beginning, the friction force between the bone drill model 2 and the skeleton model 3 and at the collision point i is supposed as being a static friction force. Hence, a horizontal speed of the collision point i after the collision should be 0, that is, \vec{U}_{cτ}_{ᵢ}=0. According to the impulse theory, the total impulse can be calculated by the following formula:

\vec{P}ᵢ=Kᵢ⁻¹(\vec{U}_{cn}_{ᵢ}−\vec{V}_{cn}_{ᵢ}−\vec{V}_{cτ}_{ᵢ}) (7)

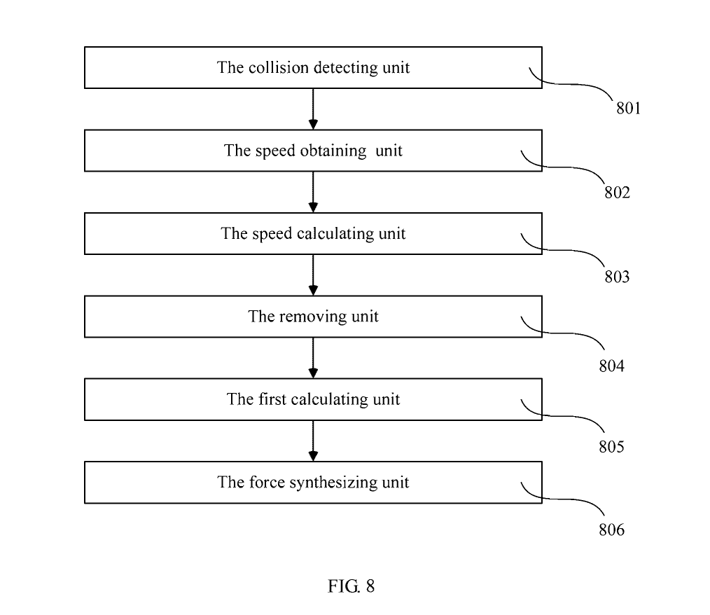

Then, whether a direction of \vec{P}ᵢ is within the friction cone or not is detected, as shown in FIG. 6.

If \vec{P}ᵢ is within the friction cone, in other words, a formula of |(\vec{P}ᵢ−\vec{P}ₙ_{ᵢ})|≦|μₛ\vec{P}ₙ_{ᵢ}|, (\vec{P}ₙ_{ᵢ}=(\vec{P}ᵢ□\vec{n}ᵢ)\vec{n}ᵢ) can be met, a state of the friction force can be regarded as being the static friction force, and finally, the static friction force can be obtained by the following formula:

$${\vec{f}}_{{τ}_{i}} = \frac{{\vec{P}}_{i} - {\vec{P}}_{{n}_{i}}}{{t}_{1} - {t}_{0}} \qquad ( 8 )$$

When \vec{P}ᵢ is not within the friction cone, it needs to be considered that the friction force may be the dynamic friction force. In this case, the horizontal speed of the collision point after the collision is not 0, and can't be calculated by the formula (7), \vec{P}ᵢ and \vec{P}ₙ_{ᵢ} need to be recalculated. According to the impulse theory, the impulse can be written to be the following formula:

Kᵢ\vec{P}ᵢ=\vec{U}_{cn}_{ᵢ}+\vec{U}_{cτ}_{ᵢ}−\vec{V}_{cn}_{ᵢ}−\vec{V}_{cτ}_{ᵢ} (9)

Using \vec{n}ᵢ to execute dot product at two sides of the formula (9) respectively, and then according to the Newton's impact law, we can obtain a formula in the following:

−(e−1)|\vec{V}_{cn}_{ᵢ}|=\vec{n}ᵢ^{T}Kᵢ\vec{P}ᵢ (10)

After that, according to the Coulomb's law, \vec{P}ᵢ can be expressed as \vec{P}ᵢ=\vec{P}ₙ_{ᵢ}−μₖ|\vec{P}ₙ_{ᵢ}|\vec{τ}ᵢ, \vec{τ}ᵢ is the unit vector of \vec{V}_{cτ}_{ᵢ}. In this way, \vec{P}ₙ_{ᵢ} and \vec{P}_{τ}_{ᵢ} under a sliding friction mode can be obtained respectively by the following formulas:

$$\begin{matrix}{\vec{P}}_{{n}_{i}} = \frac{- ( e + 1 ) \vert {\vec{V}}_{{n}_{i}} \vert {\vec{n}}_{i}}{{\vec{n}}_{i}^{T} {K}_{i} ( {\vec{n}}_{i} - {μ}_{k} {\vec{τ}}_{i} )} & ( 11 ) \\ {\vec{P}}_{{τ}_{i}} = {\vec{P}}_{i} - {\vec{P}}_{{n}_{i}} & ( 12 )\end{matrix}$$

Three, the calculation process of the resultant force after removing is as follows:

Since lots of collision points can be generated when the bone drill model 2 collides with the skeleton model 3, at the time of collision, the collision points can be arranged in a descending order according to the distances between each of the collision point pairs, and a collision point pair distance list can be generated. The collision point pair is the aforesaid discrete point and cross point corresponding to the discrete point, the distance is regarded as being a depth by which the bone drill model 2 enters the interior of the skeleton model 3 from the position of the collision; it is considered that the longer the distance, the stronger the influence on the force calculation caused by the collision taking place at the position. Then, the calculation process starts from a first collision point of the list, collision forces \vec{f}ₙ_{ᵢ}, \vec{f}_{τ}_{ᵢ} and the impulse \vec{P}ᵢ on the collision point can be obtained according to the aforesaid formulas, the movement speed \vec{U}ᵢ and the autorotation speed \vec{ω}′ of the bone drill model 2 at the collision point after the collision, and the movement speed \vec{v}ᵢ and the autorotation speed \vec{ω} of the bone drill model 2 at the collision point before the collision can be obtained based on the impulse theory, the Newton's impact law, the Coulomb's law, and according to the following formula:

\vec{U}ᵢ=\vec{V}ᵢ+\vec{P}ᵢ/M and \vec{ω}′=\vec{ω}+J⁻¹(\vec{r}ᵢ×\vec{P}ᵢ).

Thus, all collision point pairs in the list will be traversed sequentially so as to detect whether there is a relative separation speed with respect to the skeleton model, that is, whether a formula of \vec{n}ᵢ□\vec{U}_{cn}_{ᵢ}≧0 is met or not is checked; any collision point pair meeting the formula is considered as having no contribution to the entire collision, and thus it needs to be deleted from the list, until all the collision points in the list have been traversed. Then, collision forces applied on all collision points that have contribution to the collision are synthesized, and the obtained resultant force are used as a final collision contact force which is formulized as follows:

$${\vec{f}}_{c} = {\sum }_{i} ( {\vec{f}}_{{n}_{i}} + {\vec{f}}_{{τ}_{i}} ) \qquad ( 14 )$$

Four, the calculation process of the vibration force is as follows.

The bone drill model in the embodiment of the present invention comprises a round head 20, and a long shaft 21, the long shaft 21 is linked to the electric drive device. The bone drill model 2 is operated under the driving of the electric drive device, and can autorotate in a certain speed so as to grind sclerotin. Since a linking between the long shaft 21 and the electric drive device is not completely tight, and has certain looseness, such looseness may lead to certain vibrations generated when the bone drill model 2 operates. Therefore, the embodiment of the present invention has also considered the imbalance vibrations of the bone drill model 2 caused by the collision between the round head 20 and the skeleton model 3. As the imbalance vibrations caused by the collision between the round head 20 and the skeleton model 3 is a main vibration source, and can led to certain obstacle to accurate grinding, the present invention establishes a vibration model having three degrees of freedom to simulate the aforesaid imbalance vibrations; when a doctor adopts the vibration model to be trained, he/she can get a much better and more verisimilar training. As shown in FIG. 7, the long shaft 21 of the bone drill model 2 is simulated as a straight rod of which a distal end is connected to the electric drive device by a spring in an X-axis direction, a spring in a Y-axis direction and a spring in a Z-axis direction, so as to simulate vibrations in horizontal directions (the X-axis direction and the Y-axis direction) and vibrations in an axial direction (the Z-axis direction).

A vibration displacement S of the long shaft 21 of the bone drill model 2 can be obtained in a frequency domain by a transformation functional matrix Φ₍ₛ₎:

$$[ \begin{matrix}Δ x ( s ) \\ Δ y ( s ) \\ Δ z ( s )\end{matrix} ] = [ \begin{matrix}{Φ}_{xx} & {Φ}_{xy} & {Φ}_{xz} \\ {Φ}_{yx} & {Φ}_{yy} & {Φ}_{yz} \\ {Φ}_{zx} & {Φ}_{zx} & {Φ}_{zz}\end{matrix} ] [ \begin{matrix}{f}_{cx} ( s ) \\ {f}_{cy} ( s ) \\ {f}_{cz} ( s )\end{matrix} ] \qquad ( 15 )$$

Wherein [Δx(s) Δy(s) Δz(s)]^{T} is Laplace transform of the vibration displacement of the long shaft 21, [f_{cx}(s) f_{cy}(s) f_{cz}(s)]^{T} corresponds to a variable of the collision contact force between the bone drill model 2 and the skeleton model 3 in the Laplace domain. Items in the transformation functional matrix Φ(s) reflect looseness characteristics of the long shaft 21 in every direction, and can be obtained by the following formula:

$$Φ ( s ) = {\sum }_{h = 1}^{K} \frac{{ω}_{nh}^{2} \text{/} {k}_{h}}{{s}^{2} + 2 {ζ}_{h} {ω}_{nh} s + {ω}_{nh}^{2}} \qquad ( 16 )$$

Wherein ωₙₕ, kₕ and ζₕ respectively represent a nature frequency, a mode stiffness, and a damping coefficient under a mode number h.

The solution of the partial differential equation (15) can be calculated by the fourth-order Runge-Kutta numerical value method, such that the vibration displacement Δ\vec{d}(t)=[Δx(s) Δy(s) Δz(s)]^{T} can be obtained, and then the vibration force applied on the long shaft 21 can be obtained by the following formula:

\vec{f}_{vib}=2MₛΔ\vec{d}/(t₁−t₀)² (17)

Wherein, Mₛ is the mass of the long shaft 21.

In the embodiment of the present invention, if vibration forces are considered, when the resultant force is finally calculated, the resistances and the friction forces of all collision points that are not removed and the vibration force applied on the long shaft 21 are synthesized into a resultant force.

Five, the calculation process of the material attributes is as follows.

In the calculation process of the resistance force and the friction force, the hybrid coefficient of restitution e has been used, and the hybrid coefficient of restitution e represents the material properties of the bone drill model 2 and the skeleton model 3. The hybrid coefficient of restitution e is a measurement for elastic properties between colliding objects, reflects kinetic energy loss in the process of the collision, and can be obtained by the following formula:

$$e = \sqrt{( \frac{{e}_{tool}^{2} ( 1 - {v}_{tool}^{2} )}{{E}_{tool}} + \frac{{e}_{bone}^{2} ( 1 - {v}_{bone}^{2} )}{{E}_{bone}} ) E} \\ E = {E}_{tool} {E}_{bone} \text{/} ( {E}_{tool} + {E}_{bone} ) \qquad ( 18 )$$

wherein, Eₜₒₒₗ and E_{bone} are respectively Young's moduli of the bone drill and the skeleton, vₜₒₒₗ and v_{bone} are respectively Poisson's ratios of the bone drill, eₜₒₒₗ and e_{bone} are respectively coefficients of restitution of the bone drill and the skeleton, and E is an effective Young's moduli in the process of collision.

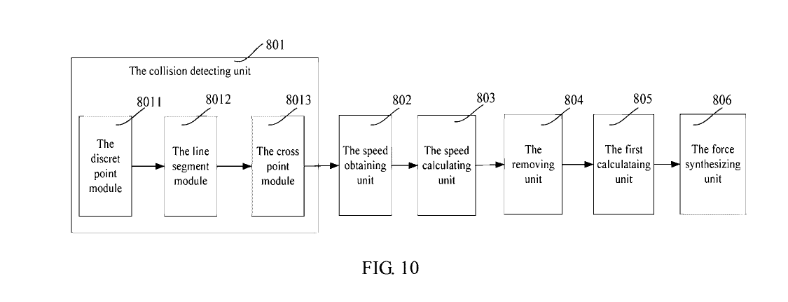

Please refer to FIG. 8, the embodiments of the present invention further provide an apparatus for simulating force interation between a bone drill and a skeleton, the apparatus comprises:

a collision detecting unit 801 configured for detecting whether a collision takes place between a bone drill model and a skeleton model in real time;

a speed obtaining unit 802 configured for: when the collision takes place, obtaining a movement speed and an autorotation speed of each collision point before the collision;

a speed calculating unit 803 configured for calculating the movement speed and the autorotation speed of each collision point after the collision according to the impulse theory, the Newton's impact law, the Coulomb's law, and the movement speed and the autorotation speed of each collision point before the collision;

a removing unit 804 configured for removing a collision point having a separation speed with respect to the skeleton model 3 after the collision;

a first calculating unit 804 configured for calculating a resistance force and a friction force on a collision point that is not removed at the time of collision according to movement speeds and autorotation speeds before and after the collision, and by a method based on the impulse theory; and

a force synthesizing unit 804 configured for synthesizing resistance forces and friction forces of all the collision points that are not removed into a resultant force to output the resultant force to a force feedback device.

Please refer to FIG. 9, in the embodiment of the present invention, the bone drill model 2 comprises a round head 20 and a long shaft 21; the apparatus further comprises:

a vibration simulating unit 807 configured for simulating the long shaft 21 as a straight rod of which a distal end is connected to an electric drive device by a spring in an X-axis direction, a spring in a Y-axis direction and a spring in a Z-axis direction respectively to simulate vibrations of the long shaft 21 in a horizontal direction and in an axial direction;

a second calculating unit 808 configured for calculating vibration forces applied on the long shaft 21 in the process of vibration;

the force synthesizing unit 806 is specifically configured for synthesizing the resistance forces and the friction forces of all the collision points that are not removed, and the vibration forces applied on the long shaft 21 into a resultant force to output the resultant force to the force feedback device.

Please refer to FIG. 10, in the embodiment of the present invention, the collision detecting unit 801 comprises:

a discrete point module 8011 configured for uniformly distributing a predefined number of discrete points in advance on a cutting-edge of the bone drill model 2;

a line segment module 8013 configured for connecting a line segment between each of the discrete points and a center point of the bone drill model;

a cross point detecting module 8013 configured for detecting whether the line segment and a triangle surface patch of the skeleton model share a cross point or not in real time; if yes, determining that the collision has taken place between the bone drill model 2 and the skeleton model 3, and recording the discrete point at which the collision has taken place as the collision point; if no, determining that the collision doesn't take place between the bone drill model 2 and the skeleton model 3;

the speed obtaining unit 802 is configured specifically for when the collision has taken place, obtaining the movement speed and the autorotation speed of each discrete point at which the collision has taken place before the collision;

the speed calculating unit 803 configured for calculating the movement speed and the autorotation speed of each discrete point at which the collision has taken place after the collision according to the impulse theory, the Newton's impact theory, the Coulomb's law, and the movement speed and the autorotation speed of each discrete point at which the collision has taken place before the collision;

the removing unit 804 is configured specifically for removing a discrete point that has been collided and having a separation speed with respect to the skeleton model 3;

the first calculating unit 805 is configured specifically for calculating a resistance force and a friction force applied on a discrete point that has been collided and is not removed according to the movement speeds and the autorotation speeds before and after the collision and by a method based on the impulse theory;

the force synthesizing unit 806 is configured specifically for synthesizing resistance forces and friction forces of all the discrete points that have been collided and are not removed into the resultant force to output the resultant force to the force feedback device. In the embodiment of the present invention, the resistance force is

$${\vec{f}}_{{n}_{i}} = \frac{{\vec{P}}_{{n}_{i}}}{{t}_{1} - {t}_{0}} ;$$

the friction force comprises a static friction force and a dynamic friction force; the static friction force is

$${\vec{f}}_{{τ}_{i}} = \frac{{\vec{P}}_{i} - {\vec{P}}_{{n}_{i}}}{{t}_{1} - {t}_{0}} ;$$

the dynamic friction force is

$${\vec{f}}_{{τ}_{i}} = \frac{{\vec{P}}_{i} - {\vec{P}}_{{n}_{i}}}{{t}_{1} - {t}_{0}} ;$$

the vibration force is \vec{f}_{vib}=2MₛΔ\vec{d}(t)/(t₁−t₀)²; a hybrid coefficient of restitution is

$$e = \sqrt{( \frac{{e}_{tool}^{2} ( 1 - {v}_{tool}^{2} )}{{E}_{tool}} + \frac{{e}_{bone}^{2} ( 1 - {v}_{bone}^{2} )}{{E}_{bone}} ) E} ,$$

wherein, E=EₜₒₒₗE_{bone}/(Eₜₒₒₗ+E_{bone});

wherein, a unit vector directed from the discrete point at which the collision has taken place to a corresponding cross point is recorded as \vec{n}ᵢ; \vec{P}ₙ_{ᵢ} is an impulse generated in the direction of the unit vector \vec{n}ᵢ of the discrete point at which the collision has taken place and in a time interval from a time t₀ to a time t₁ by the resistance force \vec{f}ₙ_{ᵢ}, \vec{P}ᵢ is a total impulse applied on the discrete point at which the collision has taken place during the process of collision lasting from the time t₀ to the time t₁; \vec{P}ₙ_{ᵢ} and \vec{P}ᵢ are calculated specifically according to the obtained movement speeds and autorotation speeds of the discrete point at which the collision has taken place before and after the collision, and according to the Newton's collision law, the impulse theory and the theorem of momentum; M, represents a mass of the long shaft, Δ\vec{d}(t) represents a vibration displacement, Δ\vec{d}(t) is calculated specifically by a transformation function matrix and a fourth-order Runge-Kutta numerical value method; Eₜₒₒₗ and E_{bone} are respectively Young's modulus of the bone drill model and the skeleton model, vₜₒₒₗ and v_{bone} are respectively Poisson's coefficients of the bone drill model and the skeleton model, eₜₒₒₗ and e_{bone} are respectively coefficients of restitution of the bone drill model and the skeleton model, and E is an effective Young's modulus in the process of collision.

The details of the scheme in the apparatus have been described in the aforesaid method, and are not repeated herein.

The embodiments of the present invention further provide a VR (Virtual Reality) surgical system, the VR surgical system comprises the aforesaid apparatus for simulating force interaction between a bone drill and a skeleton, on one hand, the VR surgical system can simulate an actual surgery operation environment as realistic as possible, such that a doctor can touch and feel a virtual patient model through the force feedback device, and exercise the capability of cooperation and coordination of the hands and the eyes in the force interaction process; on the other hand, visual scenes that show an experienced doctor operating surgical tools, movements of the hands of the doctor, and force applying processes of the doctor can be recorded and used as training courses, in this way, real surgery scenes can be reappeared and provided to young doctors for study. The VR surgical system is, in particular, suitable for training of a surgical technique which needs to be determined by a doctor according to force sensed during tool interactions completely when the vision of the doctor is restrained, this makes surgical techniques that can only be aware of in the training of surgery become being able to be personally experienced, so that the period of training and learning can be shortened.

In the method, apparatus, and system for simulating force interaction between a bone drill and a skeleton provided by the embodiment of the present invention, by using the method based on the impulse theory to calculate the resistance force and the friction force applied on each of the collision points that are not removed at the time of collision, the hybrid coefficient of restitution e can be fully reflected in the resistance forces and the friction forces, thereby well reflecting the material properties of the bone drill and the skeleton, and force differences brought by force interaction, such as grinding between the bone drill and the skeleton of different materials, can be effectively reflected. When sclerotin materials with different properties are used, or in grinding processes using bone drills of different materials, force sensing having distinct difference can be feeled. By simulating the vibrations in the X-axis direction, the Y-axis direction, and the Z-axis direction, horizontal and axial vibration forces applied on the long shaft 21 of the bone drill during the process of bone grinding can be effectively simulated, the simulation of vibrations is very helpful for a trainer to take control of errors and risks caused by rough vibrations of the bone drill, so that the range and the depth of bone grinding can be controlled much better. A realistic force interaction feedback and an interactive experience with much feeling of immersion can be provided to the doctor, such that the surgical skill of the doctor can be effectively improved, the training cost of the doctor can be reduced, and the risk of surgery to be suffered by the patient can be reduced. The present invention has also solved the problem that with respect to sclerotin materials with different properties, or in bone grinding processes using bone drills of different materials, force sensing having distinct difference can be felt, and influence on the grinding force caused by the autorotation speed of the bone drill itself can be reflected; when the autorotation speed is faster, a slighter force sensing can be felt, and when the autorotation speed is slower, a stronger force sensing can be felt, which completely complies with the effect on force sensing caused by the autorotation speed in the real world. The present invention is based on a physical collision analysis, parameters used by the present invention have clear physical attributes, and there is no need to carry out an additional and complex experical parameter measurement. The present invention can not only provide accurate force sensing experience, but also simultaneously meet the requirement of strict real-time performance of force interaction.

Experiment and Result:

In allusion to the present invention, a force sensing comparison experiment is also carried out, and the change of force sensing numerical values under the circumstances of variable movement speeds, variable autorotation speeds, variable hybrid coefficients of restitution, and variable friction coefficients are detected respectively. According to the experiment results, a following conclusion can be obtained: when the bone drill contacts the skeleton, the greater the movement speed of the bone drill, the greater the collision contact force and the vibration force felt by the user grasping the bone drill; and the faster the autorotation speed of the bone drill, the slighter the force sensing felt during the process of bone grindin; when the hybrid coefficient of restitution and the friction coefficient are increased, the collision contact force and the vibration force will be increased correspondingly. These results completely comply with the situation of grinding a skeleton by a bone drill in the real world. Meanwhile, in the experiment, several bond grinding tasks are designed as well, and volunteers without any experience and doctors in hospitals who are sophisticated in orthopedic surgery are invited to experience the VR surgical system provided by the present invention; in the experience process of the doctors, they are scored according to two aspects, i.e., the accuracy of completing the tasks and time spent on completion of the tasks, and a statistical analysis for the scoring results is performed using the Friedman's test; a result discloses that: the group of the volunteers without any experience has reflected an obvious learning curve in the process of repeatedly performing the tasks, as the repetition times of the tasks increase, they can accomplish the target tasks more accurately and more rapidly; however, in the group of the doctors who have proficient experiences, this learning curve doesn't exist. From the above, it can be seen that the VR surgical system of the present invention particularly fits with the veritable surgery situation, therefore, as for the doctors who have proficient experiences, they are more familiar with and easier to control the VR surgical system of the present invention.

What stated above are preferable embodiments of the present invention merely, and should not be regarded as being limitation to the present invention, any modification, equivalent replacement and improvement, which are made within the spirit and the principle of the present invention, should be included in the protection scope of the present invention.

1. A method for simulating force-sensing interaction between a bone drill and a skeleton, comprising:
detecting whether a collision takes place between a bone drill model and a skeleton model in real time;
when the collision takes place, acquiring a movement speed and an autorotation speed of each collision point before the collision;
calculating a movement speed and an autorotation speed of each collision point after the collision according to the impulse theory, the Newton's impact law, the Coulomb's law, and the movement speed and the autorotation speed of each collision point before the collision;
removing a collision point having a separation speed with respect to the skeleton model after the collision;
calculating a resistance force and a friction force applied on a collision point that is not removed at the time of collision according to movement speeds and autorotation speeds before and after the collision and by a method based on the impulse theory; and
synthesizing resistance forces and friction forces of all the collision points that are not removed into a resultant force to output the resultant force to a force feedback device.

2. The method according to claim 1, wherein, the bone drill model comprises a round head and a long shaft, the method further comprises:
simulating the long shaft as a straight rod of which a distal end is connected to an electric drive device by a spring in an X-axis direction, a spring in a Y-axis direction and a spring in a Z-axis direction respectively to simulate vibrations of the long shaft in a horizontal direction and an axial direction;
calculating vibration forces applied on the long shaft in the process of vibration;
the step of synthesizing resistance forces and friction forces of all the collision points that are not removed into the resultant force specifically includes:
synthesizing the resistance forces, and the friction forces of all the collision points that are not removed, and the vibration forces applied on the long shaft into a resultant force.

3. The method according to claim 1, wherein, the step of detecting whether the collision takes place between the bone drill model and the skeleton model in real time comprises:
uniformly distributing a predefined number of discrete points in advance on a cutting-edge of the bone drill model;
connecting a line segment between each of the discrete points and a center point of the bone drill model;
detecting whether the line segment and a triangle surface patch of the skeleton model share a cross point or not in real time; if yes, determining that the collision has taken place between the bone drill model and the skeleton model, and recording the discrete point at which the collision has taken place as the collision point, if no, determining that the collision doesn't take place between the bone drill model and the skeleton model;
in the step of when the collision takes place, acquiring a movement speed and an autorotation speed of each collision point before the collision, calculating a movement speed and an autorotation speed of each collision point after the collision according to the impulse theory, the Newton's impact law, the Coulomb's law, the movement speed and the autorotation speed of each collision point, removing a collision point having a separation speed with respect to the skeleton model after the collision, calculating a resistance force and a friction force applied on each collision point that is not removed at the time of collision according to movement speeds and autorotation speeds before and after the collision and by a method based on the impulse theory, and synthesizing resistance forces and friction forces of all the collision points that are not removed into a resultant force to output the resultant force to a force feedback device, the collision point is specifically the discrete point at which the collision has taken place.

4. The method according to claim 3, wherein, the resistance force is
$${\vec{f}}_{{n}_{i}} = \frac{{\vec{P}}_{{n}_{i}}}{{t}_{1} - {t}_{0}} ;$$
the friction force comprises a static friction force and a dynamic friction force: the static friction force is
$${\vec{f}}_{{τ}_{i}} = \frac{{\vec{P}}_{i} - {\vec{P}}_{{n}_{i}}}{{t}_{1} - {t}_{0}} ;$$
the dynamic friction force is
$${\vec{f}}_{{τ}_{i}} = \frac{{\vec{P}}_{i} - {\vec{P}}_{{n}_{i}}}{{t}_{1} - {t}_{0}} ;$$
the vibration force is \vec{f}_{vib}=2MₛΔ\vec{d}(t)/(t₁−t₀)²; a hybrid coefficient of restitution is
$$e = \sqrt{( \frac{{e}_{tool}^{2} ( 1 - {v}_{tool}^{2} )}{{E}_{tool}} + \frac{{e}_{bone}^{2} ( 1 - {v}_{bone}^{2} )}{{E}_{bone}} ) E} ,$$
wherein, E=EₜₒₒₗE_{bone}/(Eₜₒₒₗ+E_{bone});
wherein, a unit vector directed from the discrete point at which the collision has taken place to a corresponding cross point is recorded as \vec{n}ᵢ; \vec{P}ₙ_{ᵢ} is an impulse generated in the direction of the unit vector \vec{n}ᵢ of the discrete point at which the collision has taken place and in a time interval from a time t₀ to a time t₁ by the resistance force \vec{f}ₙ_{ᵢ}, \vec{P}ᵢ is a total impulse applied on the discrete point at which the collision has taken place during the process of collision lasting from the time t₀ to the time t₁; \vec{P}ₙ_{ᵢ} and \vec{P}ᵢ are calculated specifically according to the obtained movement speeds and autorotation speeds of the discrete point at which the collision has taken place before and after the collision, and according to the Newton's collision law, the impulse theory and the theorem of momentum; Mₛ represents a mass of the long shaft Δ\vec{d}(t) represents a vibration displacement, Δ\vec{d}(t) is calculated specifically by a transformation function matrix and a fourth-order Runge-Kutta numerical value method; Eₜₒₒₗ and E_{bone} are respectively Young's modulus of the bone drill model and the skeleton model, vₜₒₒₗ and v_{bone} are respectively Poisson's coefficients of the bone drill model and the skeleton model, eₜₒₒₗ and e_{bone} are respectively coefficients of restitution of the bone drill model and the skeleton model, and E is an effective Young's modulus in the process of collision.

5. The apparatus for simulating force-sensing interaction between a bone drill and a skeleton, comprising:
a collision detecting unit configured for detecting whether a collision takes place between a bone drill model and a skeleton model in real time:
a speed obtaining unit configured for: when the collision takes place, obtaining a movement speed and an autorotation speed of each collision point before the collision;
a speed calculating unit configured for calculating the movement speed and the autorotation speed of each collision point after the collision according to the impulse theory, the Newton's impact law, the Coulomb's law, and the movement speed and the autorotation speed of each collision point before the collision;
a removing unit configured for removing a collision point having a separation speed with respect to the skeleton model after the collision;
a first calculating unit configured for calculating a resistance force and a friction force on a collision point that is not removed at the time of collision according to movement speeds and autorotation speeds before and after the collision and by a method based on the impulse theory; and
a force synthesizing unit configured for synthesizing resistance forces and friction forces of all the collision points that are not removed into a resultant force to output the resultant force to a force feedback device.

6. The apparatus according to claim 5, wherein, the bone drill model comprises a round head and a long shaft; the apparatus further comprises:
a vibration simulating unit configured for simulating the long shaft as a straight rod of which a distal end is connected to an electric drive device by a spring in an X-axis direction, a spring in a Y-axis direction and a spring in a Z-axis direction respectively to simulate vibrations of the long shaft in a horizontal direction and an axial direction; and
a second calculating unit configured for calculating vibration forces applied on the long shaft in the process of vibration;
the force synthesizing unit is specifically configured for synthesizing resistance forces and friction forces of all the collision points that are not removed, and the vibration forces applied on the long shaft into a resultant force to output the resultant force to the force feedback device.

7. The apparatus according to claim 5, wherein, the collision detecting unit comprises:
a discrete point module configured for uniformly distributing a predefined number of discrete points in advance on a cutting-edge of the bone drill model;
a line segment module configured for connecting a line segment between each of the discrete points and a center point of the bone drill model; and
a cross point detecting module configured for detecting whether the line segment and a triangle surface patch of the skeleton model share a cross point or not in real time; if yes, determining that the collision has taken place between the bone drill model and the skeleton model, and recording the discrete point at which the collision has taken place as the collision point; if no, determining that the collision doesn't take place between the bone drill model and the skeleton model;
the speed obtaining unit is configured specifically for when the collision takes place, obtaining the movement speed and the autorotation speed of each discrete point at which the collision has taken place before the collision;
the speed calculating unit configured for calculating the movement speed and the autorotation speed of each discrete point at which the collision has taken place after the collision according to the impulse theory, the Newton's impact theory, the Coulomb's law, the movement speed and the autorotation speed of each discrete point at which the collision has taken place before the collision;
the removing unit is configured specifically for removing a discrete point at which the collision has taken place and having a separation speed with respect to the skeleton model;
the first calculating unit is configured specifically for calculating a resistance force and a friction force applied on a discrete point that has been collided and is not removed according to the movement speeds and the autorotation speeds before and after the collision and by a method based on the impulse theory;
the force synthesizing unit is configured specifically for synthesizing resistance forces and friction forces of all the discrete points that have been collided and are not removed into the resultant force to output the resultant force to the force feedback device.

8. The apparatus according to claim 7, wherein, the resistance force is
$${\vec{f}}_{{n}_{i}} = \frac{{\vec{P}}_{{n}_{i}}}{{t}_{1} - {t}_{0}} ;$$
the friction force comprises a static friction force and a dynamic friction force: the static friction force is
$${\vec{f}}_{{τ}_{i}} = \frac{{\vec{P}}_{i} - {\vec{P}}_{{n}_{i}}}{{t}_{1} - {t}_{0}} ;$$
the dynamic friction force is
$${\vec{f}}_{{τ}_{i}} = \frac{{\vec{P}}_{i} - {\vec{P}}_{{n}_{i}}}{{t}_{1} - {t}_{0}} ;$$
the vibration force is \vec{f}_{vib}=2MₛΔ\vec{d}(t)/(t₁−t₀)²; a hybrid coefficient of restitution is
$$e = \sqrt{( \frac{{e}_{tool}^{2} ( 1 - {v}_{tool}^{2} )}{{E}_{tool}} + \frac{{e}_{bone}^{2} ( 1 - {v}_{bone}^{2} )}{{E}_{bone}} ) E} ,$$
wherein E=EₜₒₒₗE_{bone}/(Eₜₒₒₗ+E_{bone});
wherein, a unit vector directed from the discrete point at which the collision has taken place to a corresponding cross point is recorded as \vec{n}ᵢ; \vec{P}ₙ_{ᵢ} is an impulse generated in the direction of the unit vector \vec{n}ᵢ of the discrete point at which the collision has taken place and in a time interval from a time t₀ to a time t₁ by the resistance force \vec{f}ₙ_{ᵢ}, \vec{P}ᵢ is a total impulse applied on the discrete point at which the collision has taken place during the process of collision lasting from the time t₀ to the time t₁; \vec{P}ₙ_{ᵢ} and \vec{P}ᵢ are calculated specifically according to the obtained movement speeds and autorotation speeds of the discrete point at which the collision has taken place before and after the collision, and according to the Newton's collision law, the impulse theory and the theorem of momentum; Mₛ represents a mass of the long shaft, Δ\vec{d}(t) represents a vibration displacement, Δ\vec{d}(t) is calculated specifically by a transformation function matrix and a fourth-order Runge-Kutta numerical value method; Eₜₒₒₗ and E_{bone} are respectively Young's modulus of the bone drill model and the skeleton model, vₜₒₒₗ and v_{bone} are respectively Poisson's coefficients of the bone drill model and the skeleton model, eₜₒₒₗ and e_{bone} are respectively coefficients of restitution of the bone drill model and the skeleton model, and E is an effective Young's modulus in the process of collision.

9. A virtual surgical system, wherein, the virtual surgical system comprises the apparatus for simulating force-sensing interaction between the bone drill and the skeleton according to claim 5.

10. The method according to claim 2, wherein, the step of detecting whether the collision takes place between the bone drill model and the skeleton model in real time comprises:
uniformly distributing a predefined number of discrete points in advance on a cutting-edge of the bone drill model;
connecting a line segment between each of the discrete points and a center point of the bone drill model;
detecting whether the line segment and a triangle surface patch of the skeleton model share a cross point or not in real time; if yes, determining that the collision has taken place between the bone drill model and the skeleton model, and recording the discrete point at which the collision has taken place as the collision point, if no, determining that the collision doesn't take place between the bone drill model and the skeleton model;
in the step of when the collision takes place, acquiring a movement speed and an autorotation speed of each collision point before the collision, calculating a movement speed and an autorotation speed of each collision point after the collision according to the impulse theory, the Newton's impact law, the Coulomb's law, the movement speed and the autorotation speed of each collision point, removing a collision point having a separation speed with respect to the skeleton model after the collision, calculating a resistance force and a friction force applied on each collision point that is not removed at the time of collision according to movement speeds and autorotation speeds before and after the collision and by a method based on the impulse theory, and synthesizing resistance forces and friction forces of all the collision points that are not removed into a resultant force to output the resultant force to a force feedback device, the collision point is specifically the discrete point at which the collision has taken place.

11. The apparatus according to claim 6, wherein, the collision detecting unit comprises:
a discrete point module configured for uniformly distributing a predefined number of discrete points in advance on a cutting-edge of the bone drill model;
a line segment module configured for connecting a line segment between each of the discrete points and a center point of the bone drill model; and
a cross point detecting module configured for detecting whether the line segment and a triangle surface patch of the skeleton model share a cross point or not in real time; if yes, determining that the collision has taken place between the bone drill model and the skeleton model, and recording the discrete point at which the collision has taken place as the collision point; if no, determining that the collision doesn't take place between the bone drill model and the skeleton model;
the speed obtaining unit is configured specifically for when the collision takes place, obtaining the movement speed and the autorotation speed of each discrete point at which the collision has taken place before the collision;
the speed calculating unit configured for calculating the movement speed and the autorotation speed of each discrete point at which the collision has taken place after the collision according to the impulse theory, the Newton's impact theory, the Coulomb's law, the movement speed and the autorotation speed of each discrete point at which the collision has taken place before the collision;
the removing unit is configured specifically for removing a discrete point at which the collision has taken place and having a separation speed with respect to the skeleton model;
the first calculating unit is configured specifically for calculating a resistance force and a friction force applied on a discrete point that has been collided and is not removed according to the movement speeds and the autorotation speeds before and after the collision and by a method based on the impulse theory;
the force synthesizing unit is configured specifically for synthesizing resistance forces and friction forces of all the discrete points that have been collided and are not removed into the resultant force to output the resultant force to the force feedback device.
